(19) United States
(12) Patent Application Publication
(10) Pub. No.: US 2017/0290940 A1
(43) Pub. Date: Oct. 12, 2017

(54) SCENT DISPENSING DEVICE

(71) Applicant: Frank E. Lee, Mesquite, NV (US)

(72) Inventor: Frank E. Lee, Mesquite, NV (US)

(21) Appl. No.: 15/096,634

(22) Filed: Apr. 12, 2016

(51) Int. Cl. A61L 9/012

(52) U.S. Cl. CPC A61L 9/012; A61L 2209/13; A61L 2209/134

(57) ABSTRACT

A scent dispensing device comprising a porous material saturated with a scent composition and dried, such that the scent composition dispenses from the porous material when air flow is induced to pass through. The porous material comprises a dispense and a mount surface. The dispense surface is covered by a cover portion minimizing contact with the scent composition. A base portion joins the mount surface. An adhesive portion on the mount surface helps adhere the porous material to an air flow inducing object that induces air flow through the porous material, dispersing the scent composition in the proximity of a path followed by the air flow inducing object. The porosity of the porous material creates turbulence that releases the scent. A panel portion covers the adhesive portion until device is ready for mounting. A tab portion provides a gripping surface for removing the panel portion.

### CROSS- REFERENCE TO RELATED APPLICATIONS

Not applicable.

### RELATED CO-PENDING U.S. PATENT APPLICATIONS

Not applicable.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to a scent dispensing device that provides a porous material saturated with a scent composition and dried, such that the scent dispenses from the porous material when air flow is induced through the porous material; whereby the device further provides an adhesive portion upon a mount surface of the porous material for selectively adhering the porous material to an air flow inducing object that enables air to flow through the porous material, and thereby disperse the scent in the proximity of path followed by the air flow inducing object.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that a scent dispenser is used to enhance the smell in a region, such as a room. The scent dispenser can use concentrated amounts of a scent composition, and then release them through various means, such as thermal energy, motion, compression, and air flow.

Typically, air fresheners can be made to attach to ceiling fan blades. These typically consists of heavy clip-on units that fasten toward the end of the blade. Such air fresheners for placement on ceiling fans are heavily encased within plastic frames. The scenting material has a few apertures through which the air can pass to circulate the desired aromas.

It is known that the particles of scent compositions are subject to gravity, including the fine mists projected by air freshening sprays. Often, the mist from an air freshener quickly falls below the elevation needed to continually enjoy its scent. An air flow can help lift the particles of the scent composition.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a perspective view of an exemplary scent dispensing device operational on an air flow inducing object and dispensing a scent composition, in accordance with an embodiment of the present invention;

FIG. 2 illustrates a top angle perspective view of an exemplary porous material from the view of a dispense surface, in accordance with an embodiment of the present invention;

FIG. 3 illustrates a bottom angle perspective view of a porous material from the view of a mount surface, in accordance with an embodiment of the present invention;

FIG. 4 illustrates a bottom angle perspective view of a scent dispensing device from a mount surface, showing an exemplary panel portion being removed from an exemplary adhesive portion in accordance with an embodiment of the present invention;

FIG. 5 illustrates a top angle perspective view of a scent dispensing device adhered to an air flow inducing object, in accordance with an embodiment of the present invention;

FIG. 6 illustrates a top angle perspective view of a scent dispensing device adhered to an air flow inducing object, and airflow flowing through a porous material to release a scent composition in accordance with an embodiment of the present invention; and

FIG. 7 illustrates a top pan view of multiple exemplary scent dispensing devices having different scent compositions, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [”substantially“] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rocklatex Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. §112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware--for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

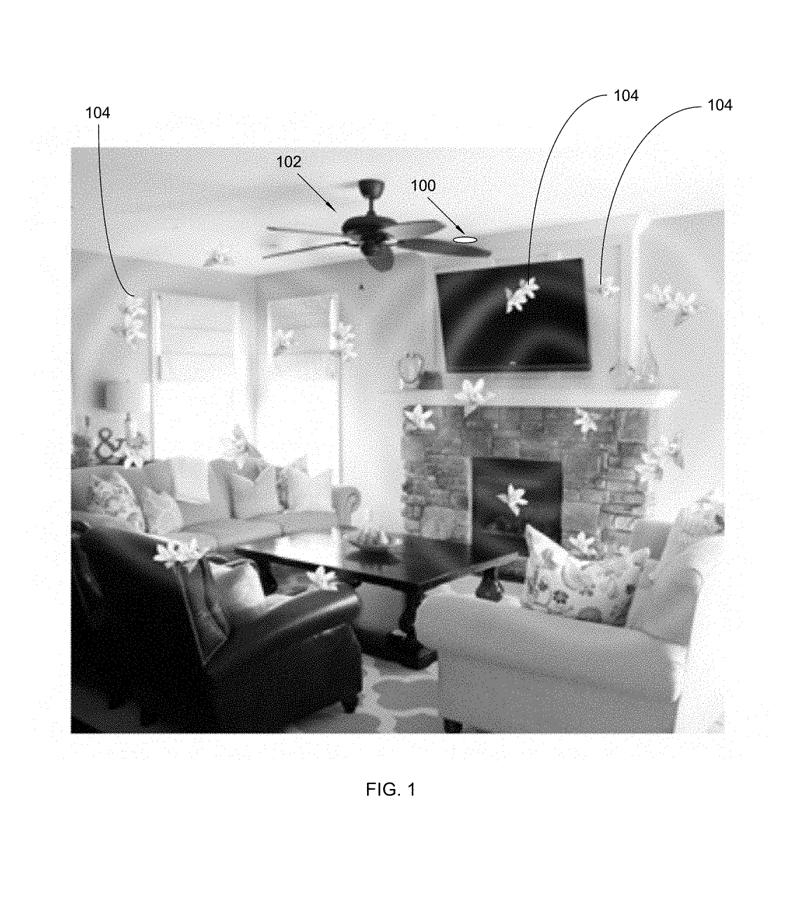

There are various types of scent dispensers that may be provided by preferred embodiments of the present invention. FIG. 1 illustrates a perspective view of an exemplary scent dispensing device 100 that is joined with an exemplary air flow inducing object 102 to release an exemplary scent composition 104. In some embodiments, the scent dispensing device may operate on the principal that a scent composition can saturate and dry in a porous material, such as foam. The generally porous characteristics of the porous material provides sufficient surface area for the scent composition to infuse and fully coat the porous material. Consequently, the fully immersed porous material may dispense the scent composition freely in a proximal area upon help from a catalyst, such as air flow, as entropic flow to least resistance, i.e., ambient air, occurs.

The porous material may receive the catalyst to release the particles of scent composition in the form of air flow passing there through. Since the porous material provides multiple channels, depressions, and cavities for the air flow, turbulent swirling action occurs near the surface of the porous material. This helps to dislodge particles of the scent composition from their infused state in the porous material to the free, ambient air proximal to the device. In some embodiments, the air flow may be brought forth by detachably joining the porous material to an air flow inducing object.

In one embodiment, the air flow inducing object moves with sufficient velocity, so as to create air flow through the porous material. In another embodiment, the air flow inducing object may include a rotating fan. Those skilled in the art will recognize that a rotating fan generates sufficient air flow to dislodge the scent particles from the porous material, but also works effectively in helping to dispense the scent composition radially, and generally uniform within the proximity of the rotating fan, i.e., a room.

In some embodiments, an adhesive portion may be used to detachably join the porous material to the air flow inducing object, and specifically a blade on the rotating fan. The adhesive material may be at least partially covered with a panel portion to prevent undesirable adherence to an object other than an air flow inducing object. The panel portion may be removed upon locating a region of the air flow inducing object to adhere the porous material. In one embodiment, a tab portion may extend from the panel portion to provide a gripping surface for removing the panel portion.

Thus, in essence, the scent dispensing device may comprise a porous material saturated with a scent composition and dried, such that the scent composition dispenses from the porous material when air flow is induced to pass through the porous material. The scent dispensing device may further provide an adhesive portion on a mount surface of the porous material for selectively adhering the porous material to an air flow inducing object that induces air flow through the porous material, and thereby disperses the scent composition in the proximity of a path that is followed by the air flow inducing object.

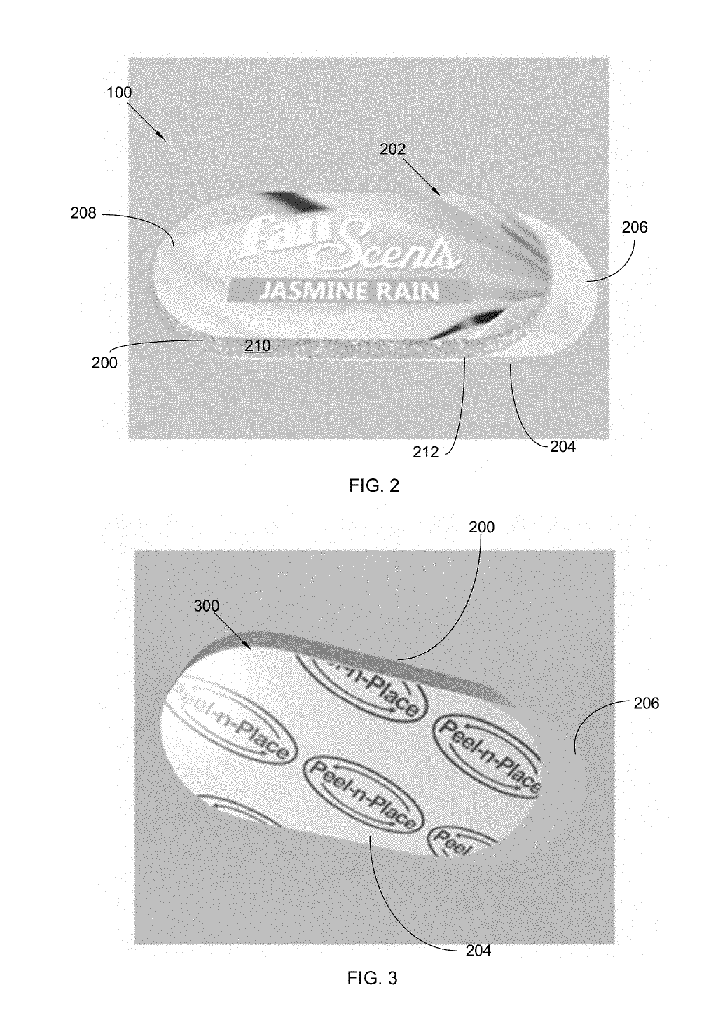

FIG. 2 illustrates a top angle perspective view of an exemplary porous material from the view of a dispense surface. In one aspect, the porous material 200 may include two faces, a dispensing surface 202 and a mount surface. The porous material may further include a sidewall 210 that positions between the surfaces. In one embodiment, the porous material may include a generally oval shape. Though other shapes may be used. In one embodiment, the porous material is about 1/4″ thick. In some embodiments, the porous material may be saturated with the scent composition, dried, and then cut into individual oval units measuring approximately two inches in length by one and one-half inches in width by one-quarter inch in height (2″×1½″×¼″)

In some embodiments, the porous material may include a piece of foam. The foam may have sufficient surface area to be saturated with the scent composition. As discussed above, the porous material also creates turbulence with the air flow, so that the scent composition may be more efficiently dislodged therefrom. Further, when the porous material is a foam, it is known that foam is sufficiently lightweight, so that a rotary fan on which the device is mounted, remains balanced and thereby provides optimal air flow through the foam.

Though in other embodiments, the porous material may include, without limitation, cork, wood, polymers, and paper products. In yet other embodiments, the porous material may include, without limitation, various foam materials that are not subject to degradation by any chemical substance, including but not limited to polyethylene, polypropylene, and polytetrafluoroethylene.

In some embodiments, a cover portion 208 may at least partially cover the dispense surface, so that the residue from the scent composition does not adhere to the hands during installation and manipulation of the device. The cover portion may include a strip of wax paper that is sized to overlay and cover a substantial portion of the dispensing surface. In other embodiments, the cover portion may include a lightweight paper that may host text and artwork covers the dispense surface.

In some embodiments, a mount surface may be oppositely disposed to the dispense surface. The mount surface may be configured to detachably join with the air flow inducing object. In some embodiments, a base portion 212 may adhere to the porous material. The porous material may adhere to the base portion. In one embodiment, the base portion may include a pliable plastic film base.

In some embodiments, a panel portion 204 may cover the base portion. The panel portion may be selectively removed from the base portion by peeling off the base portion. In some embodiments, the panel portion may include a lightweight paper that may host text and artwork covers the mount surface.

A tab portion 206 may extend from the base portion for gripping and peeling off the panel portion. The tab portion is substantially an extension of the base portion. For example, without limitation, when removing the device from a fan blade, a user may grab the tab portion and slightly pull away from the fan blade, while wiggling the tab portion. In this manipulation, the device, including the porous material and the base portion may pop off the fan blade.

In some embodiments, the tab portion may not have any adhesive or scent composition, and therefore may be safer to handle. Thus, the tab portion may be effective in ensuring that no tools, hot water or sponge are needed to remove the device from the air flow inducing object, and also ensures no adhesive residue is left on the air flow inducing object after removal therefrom.

FIG. 3 illustrates a bottom angle perspective view of a porous material from the view of a mount surface. In one aspect, the mount surface 300 may be sized and dimensioned to discretely mount the air flow inducing object. The mount surface may utilize the adhesive portion for detachably joining the air flow inducing object. The adhesive portion may utilize an adhesive.

In some embodiments, the adhesive portion may include a coating of adhesive in polyurethane gelatin format that is applied on a bottom lateral plane of the base portion, except for a small area that functions as a removal tab portion. In some embodiments, the adhesive portion may also include various adhesives of pressure-sensitive (PSA) format capable of maintaining its placement upon a ceiling fan in motion, such as but not limited to polyurethane gelatin, vinyl film of removable quality, and acrylic adhesive.

Though in other embodiments, the adhesive portion may include, without limitation, a magnet, a friction fit, a fastener, a hook and loop fastener, and a cable to securely mount the air flow inducing object. It is significant to note that since the air flow inducing object is moving at a velocity, the adhesive portion must be secure enough to retain the device on the air flow inducing object.

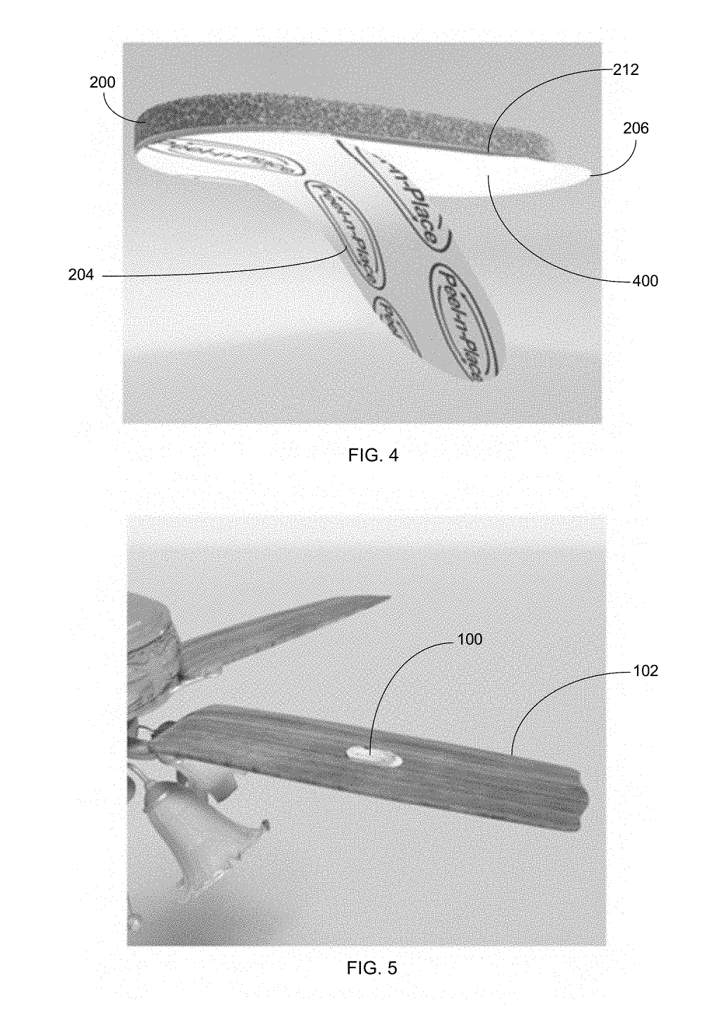

FIG. 4 illustrates a bottom angle perspective view of a scent dispensing device from a mount surface, showing an exemplary panel portion being removed from an exemplary adhesive portion. In one aspect, the device mounts to an air flow inducing object. The air flow inducing object may include a rotary fan. Though any object that induces the flow of air past the porous material and is adaptable to an adhesive portion 400 that adheres the porous material to the air inducing object may be used.

FIG. 5 illustrates a top angle perspective view of a scent dispensing device adhered to an air flow inducing object. In one aspect, the air flow inducing object moves with sufficient velocity, so as to create air flow through the porous material. In another embodiment, the air flow inducing object may include a rotating fan. Those skilled in the art will recognize that a rotating fan generates sufficient air flow to dislodge the scent particles from the porous material, but also works effectively in helping to dispense the scent composition radially, and generally uniform within the proximity of the rotating fan, i.e., a room.

In one embodiment, the device may be mounted on a top side of a blade of a rotary fan, as illustrated in FIG. 5. The device may also be placed generally in a central area of the fan to maintain balance for the rotary fan. Further, because the adhesive portion may include a thin strip of adhesive, rather than a large, bulky fastening mechanism, the rotary fan remains balanced, so as to create a uniform air flow.

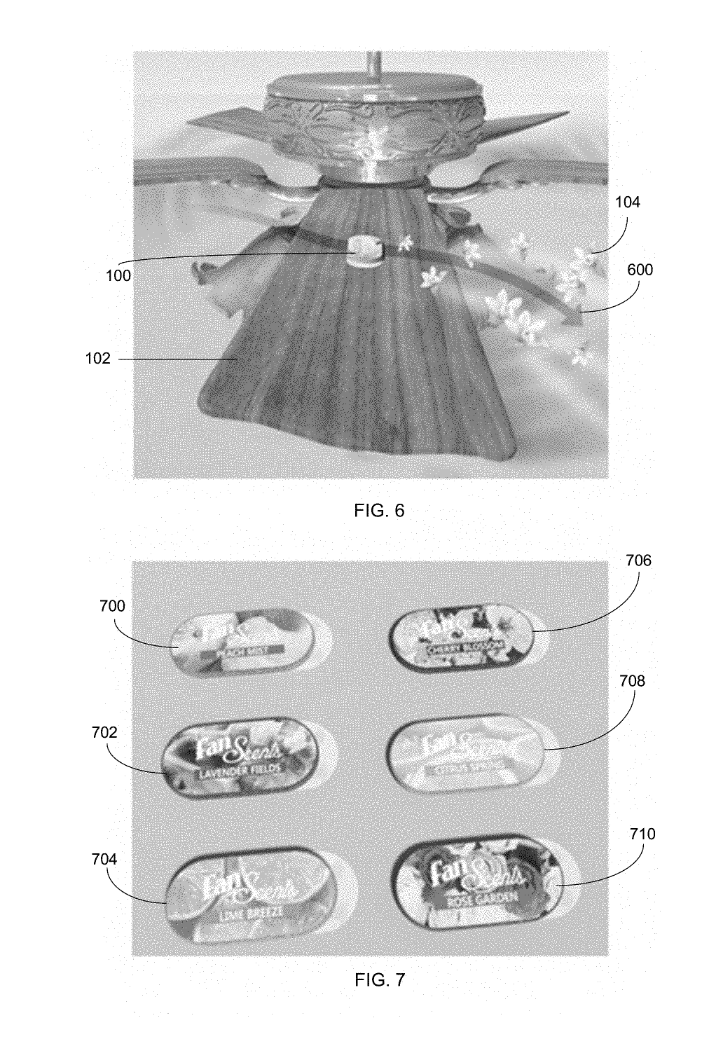

FIG. 6 illustrates a top angle perspective view of a scent dispensing device adhered to an air flow inducing object, and also showing air 600 flowing through a porous material to release a scent composition. As illustrated in FIG. 6, the rotational velocity of the air flow inducing object creates an air flow through the porous material. The air flow inducing object may include a rotary fan. The rotary fan may be especially effective since the air flow is generally uniform and disperses downwardly towards the ground, where people often congregate.

FIG. 7 illustrates a top pan view of multiple exemplary scent dispensing devices having different scent compositions. The scent dispensing device may utilize a variety of scent compositions. Though the operating principal is generally the same despite the scent. The various scent compositions may include, without limitation, a peach mist scent composition 702, a lavender fields scent composition 704, a lime breeze scent composition 706, a cherry blossom scent composition 708, a citrus spring scent composition 710, and a rose garden scent composition 712. Though other scent compositions may also be used. In any case, the scent composition saturates the porous material, and then is left to dry for a period before being dispensed through the aforementioned air flow.

In one alternative embodiment, the scent dispensing device is built into a rotary fan and does not require attachment. In another alternative embodiment, the air flow inducing device may be stationary, such as an air conditioner; whereby the device adheres to the vents of the air conditioner. In another alternative embodiment, the adhesive portion is magnetic.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing a scent dispensing device having a porous material saturated with a scent composition and dried, such that the scent composition dispenses from the porous material when air flow is induced to pass through the porous material according to the present invention will be apparent to those skilled in the art. Various aspects of the invention have been described above by way of illustration, and the specific embodiments disclosed are not intended to limit the invention to the particular forms disclosed. The particular implementation of the scent dispensing device having a porous material saturated with a scent composition and dried, such that the scent composition dispenses from the porous material when air flow is induced to pass through the porous material may vary depending upon the particular context or application. By way of example, and not limitation, the scent dispensing device utilizes a foam that has been saturated with a scent composition and mounted on a rotary fan to induce air flow through the pores of the porous material for dispensing the scent composition in the path of the rotary fan implementations; however, similar techniques may instead be applied to an air conditioner, whereby the scent inducing device attaches to the vents, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

What is claimed is:

1. A device comprising:
a porous material, said porous material comprising a scent composition, said porous material being configured to dispense said scent composition upon being induced by an air flow, said porous material further comprising a dispense surface, a mount surface, and a sidewall;
a base portion, said base portion being configured to join with said mount surface of said porous material, said base portion comprising a generally thin, pliable configuration;
a cover portion, said cover portion being configured to at least partially overlay said dispense surface of said porous material, said cover portion further being configured to help inhibit contact with said scent composition;
an adhesive portion, said adhesive portion being configured to join with said base portion, said adhesive portion further being configured to enable mounting of said device to an air flow inducing object;
a panel portion, said panel portion being configured to at least partially overlay said adhesive portion and said base portion; and
a tab portion, said tab portion being configured to extend from said base portion.

2. The device of claim 1, wherein said porous material comprises foam.

3. The device of claim 2, wherein said foam comprises at least one of the following:
polyethylene, polypropylene, and polytetrafluoroethylene.

4. The device of claim 3, wherein said porous material is approximately ¼ inches thick.

5. The device of claim 4, wherein said porous material is approximately two inches in length by one and one-half inches in width by one-quarter inch in height.

6. The device of claim 5, wherein said porous material has a generally oval shape.

7. The device of claim 6, wherein said porous material is immersed and saturated with said scent composition, and then dried.

8. The device of claim 7, wherein said cover portion comprises a strip of wax paper.

9. The device of claim 8, wherein said adhesive portion includes at least one member selected from the group consisting of: a coating of adhesive in polyurethane gelatin format, a vinyl film of removable quality, and acrylic adhesive.

10. The device of claim 9, wherein said cover portion comprises text.

11. The device of claim 10, wherein said panel portion comprises text.

12. The device of claim 11, wherein said adhesive portion is configured to be applied to said mount surface.

13. The device of claim 12, wherein said base portion comprises a pliable film base that is substantially coated with the adhesive portion.

14. The device of claim 13, wherein said tab portion is generally circular shaped.

15. The device of claim 14, wherein said air flow inducing object comprises a rotary fan.

16. The device of claim 15, wherein said rotary fan comprises a plurality of blades.

17. The device of claim 16, wherein said device is configured to mount on a top side of said plurality of blades.

18. The device of claim 17, wherein said scent composition includes at least one member selected from the group consisting of: a peach mist scent composition, a lavender fields scent composition, a lime breeze scent composition, a cherry blossom scent composition, a citrus spring scent composition, and a rose garden scent composition.

19. A device comprising:
means for saturating a dispensing means with a scenting means;
means for drying said dispensing means;
means for mounting said dispensing means;
means for inducing air flow through said dispensing means; and
means for dispensing said scenting means with the air flow inducing means.

20. A device consisting of:
a porous material, said porous material comprising a foam, said porous material further comprising a scent composition, said scent composition including at least one member selected from the group consisting of: a peach mist scent composition, a lavender fields scent composition, a lime breeze scent composition, a cherry blossom scent composition, a citrus spring scent composition, and a rose garden scent composition, said porous material being configured to dispense said scent composition upon being induced by an air flow, said porous material further comprising a dispense surface, a mount surface, and a sidewall;
a base portion, said base portion being configured to join with said mount surface of said porous material, said base portion comprising a generally thin, pliable configuration;
a cover portion, said cover portion being configured to at least partially overlay said dispense surface of said porous material, said cover portion further being configured to help inhibit contact with said scent composition;
an adhesive portion, said adhesive portion being configured to join with said base portion, said adhesive portion further being configured to enable mounting of said device to an air flow inducing object;
a panel portion, said panel portion being configured to at least partially overlay said adhesive portion and said base portion; and
a tab portion, said tab portion comprising a wax paper strip, said tab portion being configured to extend from said base portion.
